(19) United States
(12) Patent Application Publication
(10) Pub. No.: US 2025/0214179 A1
(43) Pub. Date: Jul. 3, 2025

(54) IMPROVED PLASTIC MASKS

(71) Applicant: KAJUEM HOLDINGS PTY LIMITED, Oatley, New South Wales (AU)

(72) Inventor: Andrew WISEMAN, Oatley, New South Wales (AU)

(73) Assignee: KAJUEM HOLDINGS PTY LIMITED, Oatley, New South Wales (AU)

(21) Appl. No.: 18/851,873

(22) PCT Filed: Mar. 30, 2023
(86) PCT No.: PCT/AU2023/050235

§ 371 Date: Sep. 27, 2024

(30) Foreign Application Priority Data
Mar. 30, 2022 (AU) 2022900822

(51) Int. Cl. B23K 26/382; A61M 16/06; B23K 26/0622; B23K 26/08; B23K 26/402; B23K 103/00

(52) U.S. Cl. CPC B23K 26/389; A61M 16/06; B23K 26/083; B23K 26/402; A61M 2207/00; A61M 2207/10; B23K 26/0624; B23K 2103/42

(57) ABSTRACT

Disclosed herein is an apparatus for drilling an array of vents in a plastic mask. The apparatus comprises a drill; a support comprising a surface configured to receive the mask thereat; and a positioning system configured to position the support relative to the drill for drilling vents at sequential locations in the array through a mask received at the surface.

### TECHNICAL FIELD

The present invention relates to apparatus and methods for producing improved plastic masks, and particularly plastic masks such as sleep apnoea masks having improved ventilation properties.

### BACKGROUND ART

Sleep apnoea is a condition that affects many people. For example, up to 5% of Australians and around 1 in 4 men over the age of 30 years are reported to have some degree of sleep apnoea. Sleep apnoea occurs when a person's throat muscles relax too much when they are sleeping, restricting airflow into their lungs and causing them to wake up for a few seconds (often without even knowing). As these events can happen hundreds of times a night, people suffering from sleep apnoea often wake up feeling unrefreshed and suffer from fatigue and sleepiness throughout the day. Sleep apnoea can also lead onto chronic health issues and associated health risks.

One of the methods used to treat sleep apnoea is to supply air at an increased pressure to a patient's nose and/or mouth. Indeed, devices which provide a continuous positive airway pressure (commonly referred to as CPAP devices) are the most common treatment for sleep apnoea.

It has been an enduring challenge to produce masks that can provide the necessary positive air pressure to the patient's nose and/or mouth but which are comfortable enough to wear whilst sleeping. Challenges faced by CPAP mask manufacturers include comfort, ensuring an effective seal between the mask and the patient's face (in order to maintain the increased pressure), as well as providing an appropriate degree of ventilation through the mask so that the positive pressure is maintained below a threshold at which air might escape the mask via other pathways. Noise, both from the device supplying the air to the mask and caused by air as it exists the mask's vents can also detract from the usability of CPAP devices. Addressing these challenges is important because patient compliance can become problematic if their CPAP mask is uncomfortable or CPAP device too noisy.

It would be advantageous to provide new methods and apparatus which produce plastic masks that have the potential to alleviate some of the problems associated with current masks and therefore potentially improve patient compliance with use of CPAP devices, for example.

### SUMMARY OF INVENTION

In a first aspect, the present invention provides an apparatus for drilling an array of vents in plastic masks, such as sleep apnoea masks. The apparatus comprises a drill, a support comprising a surface configured to receive the mask thereat, and a positioning system configured to position the support relative to the drill for drilling vents at sequential locations in the array through a mask received at the support.

In some embodiments, the support surface may be configured to receive the mask in an airtight manner, with the apparatus further comprising a gas source configured to deliver a gas between the mask and the surface, and a sensor for measuring a change in flow of the gas through the mask as the vents are drilled. The change in flow may, for example, be a change of overall flow rate through the mask or a change in flow direction of gas through the mask (which may affect noise levels or patient comfort, for example).

The inventor has discovered a new way of providing vents in plastic masks for use with CPAP devices, for example. Conventionally, injection molding is used to produce CPAP masks, with the injection moulding tool being provided with features that result in the mask's vent holes being formed during the molding process. The holes that can be formed in masks using this technique are, however, limited in that they cannot have a size below that which can be made without the mold becoming too thin and the attendant risk of failure, and they can only be formed in one plane. The inventor also recognised that vent hole size and the consequent vent flowthrough can significantly vary between masks that are produced via injection moulding, and that changing or tuning the vent flow is complex and time consuming, often requiring iterative tooling changes. Such complications can adversely affect the consistency of hole size and consequently air flow through the plastic masks, as well as increasing the time to market and reducing production volumes for CPAP masks.

The present invention advantageously enables plastic masks, such as CPAP masks, to be molded without vent holes (in a simpler process, using simpler molds) and then, in a separate process, finished by drilling an array of vents in the mask. In some embodiments of the invention, the holes may be drilled whilst the change in vent flow through the mask is measured, which would provide real time data that enables an unprecedented accuracy in managing vent flowthrough in a CPAP mask. No trial and error and expensive retooling would be required, and there would be an almost unlimited flexibility in the configuration of the array of vents. The inventor anticipates that the present invention may even enable individual masks to be produced (perhaps even at the point of sale) to satisfy the vent flow requirements for a patient's specific diagnosis, therapy and/or CPAP device. The array of vents should impart a high degree of tolerance and, as every mask is processed to the same specification, batch variations should be eliminated.

In some embodiments, an alignment of the drill with respect to a surface of the mask may be variable. For example, the drill may be alignable with respect to the surface of the mask at an angle of between about 90° (i.e. normal to the mask's surface) and about 45°. Providing vents in the mask having different orientations with respect to each other will control the direction of vented gases, which should reduce the noise emanating from the mask in use.

In some embodiments, the positioning system may be a 5-axis positioning system. In some embodiments, the positioning system may be operable to move the support (and hence the mask) relative to the drill, which remains stationary.

In some embodiments, the drill may be a laser drill, such already being used for producing holes through materials such as plastics and metals.

In some embodiments, the drill may be configured to drill micro holes (i.e. holes that are micrometers in diameter, e.g. about 50 micron to about 300 micron). In some embodiments, the drill may be configured to drill macro holes (i.e. holes that are between about 0.5 mm and about 0.9 mm in diameter). In some embodiments, the drill may be configurable to drill both micro and macro holes. Varying the sizes of the holes throughout the array could be used to advantage, for example, in reducing noise or in directing stronger airflows in directions that are likely to be less annoying to the patient.

In some embodiments, the array may be refined during performance of the invention, with a final configuration of the array being determined by a pre-determined flow rate of the gas through the mask. In this manner, data obtained during performance of the invention provides feedback that enables the production of a mask having a bespoke vent flowrate and pattern. The inventor envisages that bespoke software applications executing on appropriate computer hardware will be able to provide this functionality.

In some embodiments, the array of vents may provide a secondary, aesthetic, function, by defining indicia (e.g. a brand name) on the mask. Whilst such an array might not necessarily provide a specific functional advantage (i.e. over an array having a similar number of vents), the marketing appeal of such cannot be understated.

In a second aspect, the present invention provides a method for drilling an array of vents in a plastic mask (e.g. a sleep apnoea mask). The method comprises sequentially positioning the mask relative to a drill whereby sequential vents in the array are drilled through the mask; and measuring a change in flow of a gas (e.g. flow rate, flow direction, etc.) through the mask as the vents are drilled.

In some embodiments, the mask may be positioned relative to the drill using a positioning system (e.g. 5-axis positioning system, as described herein in the context of the apparatus).

In some embodiments, the method may include a preliminary step in which the mask is placed on a support of the positioning system, the support having a surface configured to receive the mask in an airtight manner.

In some embodiments, the positioning system may move the mask relative to the stationary drill.

In some embodiments, the vents may be drilled through the mask in directions that vary from being normal to the mask. In some embodiments, at least some of the vents are micro holes. In some embodiments, at least some of the vents are macro holes.

In some embodiments, a measured flow rate of the gas through the mask may be determinative of a final configuration of the array.

In some embodiments, the array may define indicia, such as a brand name or a model number, on the mask.

In a third aspect, the present invention provides a plastic mask produced using the first aspect of the present invention or the method of the second aspect of the present invention.

Additional features and advantages of the various aspects of the present invention will be described below in the context of specific embodiments. It is to be appreciated, however, that such additional features may have a more general applicability in the present invention than that described in the context of these specific embodiments.

### BRIEF DESCRIPTION OF DRAWINGS

Embodiments of the present invention will be described in further detail below with reference to the following drawings, in which:

FIG. 1 shows a mask produced in accordance with an embodiment of the present invention;

FIG. 2 shows another mask produced in accordance with an embodiment of the present invention;

FIG. 3 shows another mask produced in accordance with an embodiment of the present invention;

FIG. 4 shows a positioning system for use with embodiments of the present invention; and

FIG. 5 shows an expanded view of the mask receiving surface of the positioning system of FIG. 4.

### DESCRIPTION OF EMBODIMENTS

As described above, the present invention provides an apparatus for drilling an array of vents in plastic masks (e.g. sleep apnoea masks). The apparatus comprises a drill, a support having a surface that is configured to receive the mask thereat, and a positioning system configured to position the support (and hence the mask) relative to the drill for drilling vent holes through the mask at sequential locations in the array.

The present invention also provides a method for drilling an array of vents in a plastic mask (e.g. a sleep apnoea mask). The method comprises sequentially positioning the mask relative to a drill whereby sequential vents in the array are drilled through the mask, and measuring a change in flow of a gas (e.g. flow rate, flow direction, etc.) through the mask as the vents are drilled.

The plastic masks described herein are primarily in the form of sleep apnoea masks for use with CPAP devices and therapies. It is to be appreciated, however, that the present invention is more generally applicable and may be performed on any plastic masks that require holes to be drilled in them in order to provide their required functionality (e.g. masks used during ventilation or treatment of chronic obstructive pulmonary disease COPD).

The vented masks produced in accordance with the present invention may be produced from plastic masks having a wide variety of configurations and formed from a wide variety of plastic materials. The inventor envisages that practically any plastic mask could be finished using the present invention, suitably adapted in light of the nature of the plastic material, the mask's shape and configuration, and its intended functionality.

Performance of the invention results in the plastic mask having an array of vents. The array may be characterised by factors including the number of holes, their size and direction (i.e. the angle at which they extend through the mask), as well as by their pattern on the mask. The array of vents may have any configuration that results in the mask meeting its functional requirements, although an aesthetic appeal may also be imparted. For example, the array may have a shape and configuration that defines an indicia (e.g. the logo of the mask supplier) on the mask.

The characteristics of the array may, in some embodiments, be pre-defined. For example, a computer program may control movement of components of the apparatus (i.e. as described below) such that sequential holes of an array having a predetermined configuration are drilled in the mask.

Alternatively, in more specific embodiments, a final configuration of the array may be determined by parameters measured during performance of the invention, such as a real-time flow rate of a gas through the vents already drilled in the mask. In such embodiments (described in further detail below), a bespoke array of drilled vents may be created based on measured parameters such as gas flow through the mask, a noise level and/or a desired aesthetic outcome. In such embodiments, masks having an extremely tight tolerance range and high level of consistency between masks should be able to be produced. For example, real time vent feedback information would result in each and every mask being produced having a precise (and substantially identical) flowthrough.

In order to provide such functionality, the surface of the mask receiving support may be configured to receive the mask in an airtight manner. The apparatus may also further comprise a gas source that is configured to deliver a gas to the volume between the mask and the mask receiving support, as well as a sensor to measure a change in flow of the gas through the mask as the vents are drilled. Such an apparatus would enable the real-time measurement of the mask's ventilation properties as successive vent holes are drilled therethrough.

In a specific embodiment, for example, a custom fixture that holds the mask may be mounted to the positioning system. Connections may be provided with the fixture to enable it to be connected to a flow testing device, thereby allowing for the real time feedback whilst drilling is happening. The inventor envisages that gas flow rate monitors such as those sold by Furness Controls Limited (UK) could be used in this assembly.

Measuring a change in flow of the gas through the mask as the vents are drilled may, for example, involve the measurement of a change of overall flow rate through the mask, or a change in flow direction of gas through the mask (which may affect noise levels).

A sensor in the form of a flow meter that can output data electronically may be used. The inventor notes that it may also be useful to incorporate a smoke producing apparatus in order to visually examine the flow through the vent and above the vent (i.e. once the flow has exited the mask via the vent) to determine laminar or turbulent flow, and see how this changes when the parameters described above are varied. Cameras may also be useful to capture these data in slow motion for subsequent analysis.

The apparatus of the present invention comprises a drill. Any suitable drill may be used, including mechanical, water and laser drills. Laser drills, such as a nanosecond, picosecond or femtosecond devices, or waterjet style devices are envisaged. The inventor notes that picosecond and femtosecond UV lasers are currently used to machine plastics to avoid heat damage and would thus likely be useful in the present invention.

In some embodiments, an alignment of the drill with respect to a surface of the mask may be variable (e.g. the drill may be alignable with respect to a surface of the mask at an angle of between about 90° and 45°). The inventor expects that holes drilled through the mask at different angles will allow for better air dispersion, and hence possibly less noise than is the case for conventional masks.

The size of the holes the drill is configured to drill may be micro holes (e.g. holes that are micrometers in diameter, starting from about 2 microns and more preferably between about 50 micron and about 300 micron) and/or macro holes (e.g. holes that are closer to a millimeter in diameter, e.g. up to about 0.9 mm in diameter, or between about 0.5 mm and about 0.8 mm or about 0.9 mm in diameter). Combinations of such holes are expected to result in masks that have unique and highly functional properties. For example, the inventor expects that an array of 0.4 mm holes could produce a vent that looks like a manufacturer's logo on the side, or both sides, of a mask frame, or an insert that is in the frame.

The inventor also expects that the present invention will enable vents to be incorporated into positions in masks that were previously not possible due to the mask's geometry and manufacturing techniques. Such a combination of features should enable the production of masks having smaller, and a greater number of, vent holes. Alternately, this should allow for similarly sized holes as are currently produced in masks (i.e. using injection molding) but which are controlled to a much tighter tolerance.

The apparatus of the present invention also comprises a positioning system configured to position the mask relative to the drill for drilling holes through the mask at sequential locations in the array.

Any positioning system capable of manipulating the support upon which the mask is received may be used in the present invention. Suitable positioning systems envisaged to be useful include 5-axis positioning systems or 6-axis positioning systems. Such systems may, for example, include a multi axis table which is moveable to allow the plastic mask to be presented to the drill in a wide variety of relative orientations. Commercially available examples of such positioning systems include Stewart platforms and Hexapod Platforms, which the inventor believes will be adaptable for use in the present invention. A 6-axis system may include a commercially available 6-axis robot.

In some embodiments, the positioning system may be operable to move the support (and hence the mask) relative to the drill. In some embodiments, the positioning system may be operable to move the drill relative to the support.

The inventor envisages that software applications executing on appropriate computer hardware will be operable to control the respective parts of the apparatus in the required manner, operating in both “predetermined” and “real-time” modes. It is envisaged that the software will be capable of learning from extensive testing of different hole sizes resulting in optimisation of flow, noise and other parameters.

Such an algorithm may enable a customer to specify a vent condition in Litres/minute (e.g. between about 30 L/min and about 60 L/min, as determined by the mask design) at a pressure that will determine the configuration of the array of vent holes required. Such an algorithm may also determine a way of producing the array of holes on a 3D surface, using 3D software such as Solidworks or Autocad, with the data being exported to the drilling machine.

The prototyping conducted thus far by the inventor has produced plastic materials having an array of holes drilled therethrough using a high energy femtosecond and UV nanosecond laser. These initial trials have established proof of concept and the inventor will now adapt the established protocols to produce masks in accordance with present invention using, for example, the embodiment described below.

A conventional mask or mask insert is produced without vent holes, using any suitable technique. The mask or insert is then positioned on a receiving surface of the apparatus' positioning system, where an airtight seal is formed between the surface and the mask (much as would be the case when a patient wears the mask). A computer system that operates the apparatus and method, and which has been programmed with the appropriate mask pattern, is then activated, along with a flow of gas into the mask (this may not be caused to occur until at least part of the array of vent holed has been drilled). Drilling subsequently commences, with the mask being moved sequentially between drilling operations in order to present the mask to the drill in an appropriate orientation for drilling the next vent hole in the array. Such drilling continues until a predetermined flow is achieved or a predetermined pattern is finished, as determined by either the flow rate or the number of holes. For example, if a customer specifies that the flow rate is critical, then only the holes required to give that flow rate need be drilled. However, if the number/configuration of holes was more critical to the customer, and they were willing to accept a wider flow tolerance, then the process would concentrate on drilling those holes.

Finally, the completed mask is unloaded from the drilling machine, cleaned and packed.

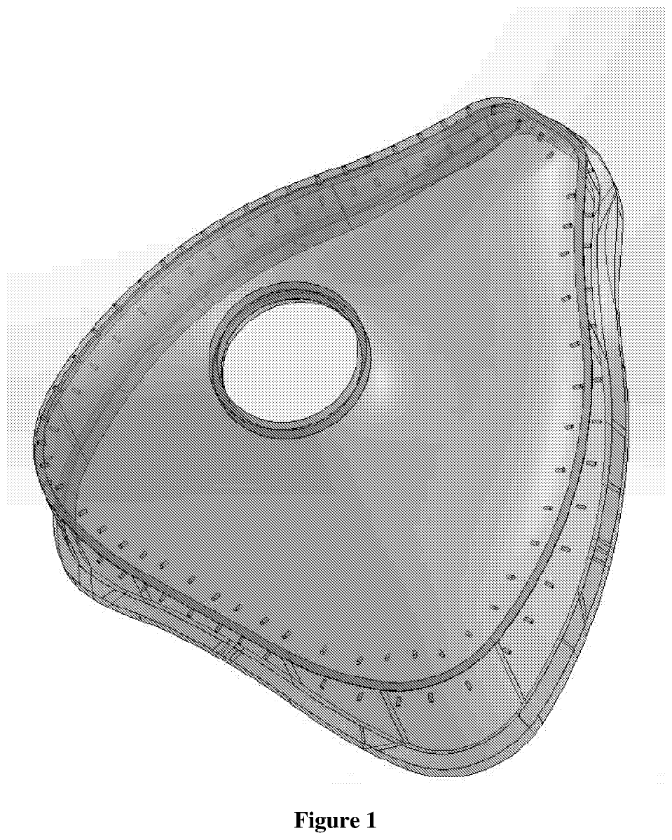

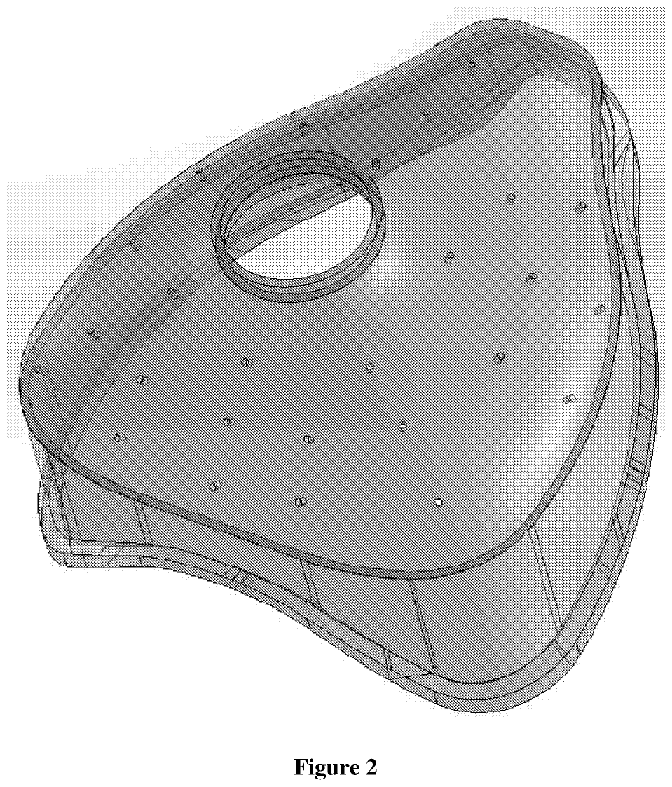

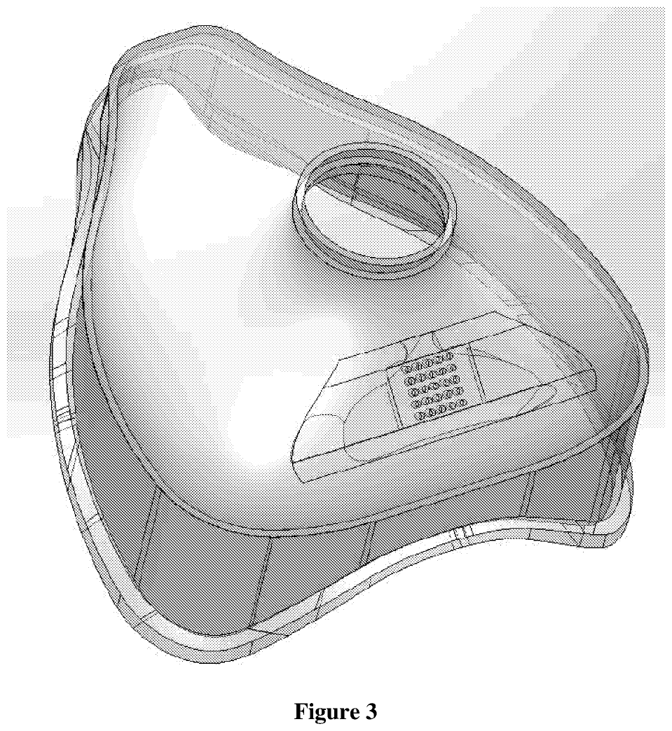

In this manner, the masks shown in FIGS. 1 to 3 can be produced. In the drawings, the masks have an array of vents around a periphery of the mask (FIG. 1), spaced about the front face of the mask (FIG. 2) and focussed proximal to the user's mouth (FIG. 3). The array of vents in the masks shown in FIGS. 1 and 2 could not be produced via injection molding and, being more diffuse vents, may provide advantages such as a lower noise profile and a tighter vent flow tolerance in use. The array of vents in the mask shown in FIG. 3 might possibly be produced via injection molding, however a more cost effective tool could be used to produce the base mask, with the array of holes subsequently drilled post molding to achieve the advantages described above.

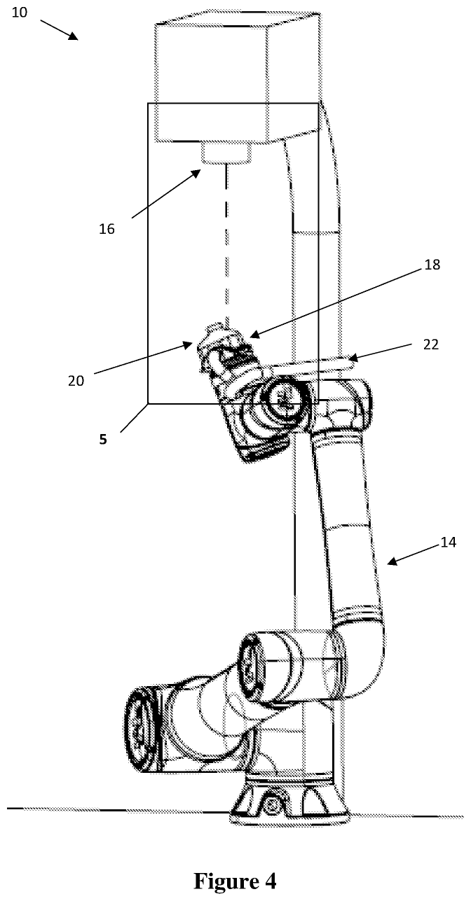

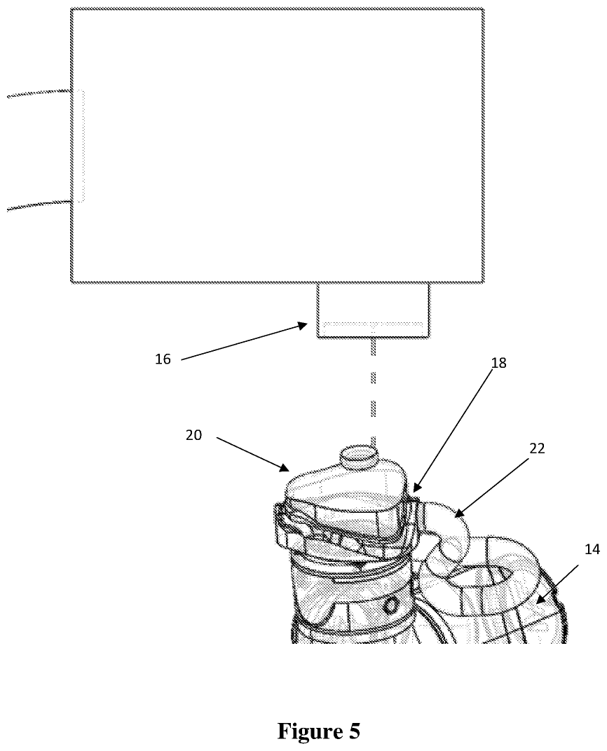

Referring finally to FIGS. 4 and 5, shown is an apparatus 10 for drilling an array of vents in a plastic mask. Apparatus 10 includes a 6-axis robot arm 14 and a laser drill 16. The distal end of arm 14 includes a support surface 18 for a mask 20, which can be moved into practically any relative configuration to drill 16 via operation of one of more of the arm's axes.

FIG. 5 shown a closer view of the distal end of arm 14, its support surface 18 and a mask 20 supported thereon. Support surface 18 is configured to sealingly receive mask 20 thereon, in essentially the same manner in which the mask would be received on a patient's face. An air hose 22 is operatively associated with the distal end of arm 14 such that air which passes through the hose 22 enters a chamber defined by surface 18 and mask 20, and subsequently comes out of the vent holes in the surface of the mask 20. The airflow through hose 22 can also be varied in order to simulates a patient breathing (e.g. in a CPAP environment). A pressure inside of the mask 20 (i.e. in the chamber defined by surface 18 and mask 20) can be monitored, as can a flow rate of air through hose 22, in order to determine a rate of airflow through the vents in the masks (i.e. as they are successively drilled).

It will be appreciated that the present invention is likely to provide a number of useful advantages over the state of the art. For example, specific embodiments of the present invention may provide one or more of the following advantages:
masks having a bespoke vent patterns and flow rate can be produced as desired, without needing new moulds etc.;
a faster and cheaper method for prototyping vents is provided, with there being no need for expensive and costly tools and subsequent tuning;
accuracy and repeatability of vent flow is significantly improved;
holes having sizes smaller and with a higher tolerance than is currently possible can be produced, providing an unparalleled versatility in adjusting vent flow rates;
a larger number of vent holes than is currently possible may be provided in medical masks; and
noise produced by the vented air can be reduced by varying the angles of the vent holes.

It will be understood to persons skilled in the art of the invention that many modifications may be made without departing from the spirit and scope of the invention. All such modifications are intended to fall within the scope of the following claims.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. An apparatus for drilling an array of vents in a plastic mask, the apparatus comprising:
a drill;
a support comprising a surface configured to receive the mask thereat; and
a positioning system configured to position the support relative to the drill for drilling vents at sequential locations in the array through a mask received at the support.

2. The apparatus of claim 1, wherein an alignment of the drill with respect to the mask is variable.

3. The apparatus of claim 2, wherein the drill is alignable with respect to a surface of the mask at an angle of between about 90° and 45°.

4. The apparatus of claim 1, wherein the positioning system is a 5-axis positioning system.

5. The apparatus of claim 1, wherein the positioning system moves the support relative to the drill.

6. The apparatus of claim 1, wherein the drill is a laser drill.

7. The apparatus of claim 1, wherein the drill is configured to drill micro holes.

8. (canceled)

9. The apparatus of claim 1, wherein the surface of the support is configured to receive the mask in an airtight manner, the apparatus further comprising:
a gas source configured to deliver a gas between the surface and the mask; and
a sensor to measure a change in flow of the gas through the mask as the vents are drilled.

10. The apparatus of claim 9, wherein a final configuration of the array of vents is determined by a measured flow rate of the gas through the mask.

11. The apparatus of claim 1, wherein the array of vents defines an indicia on the mask.

12. (canceled)

13. A method for drilling an array of vents in a plastic mask, the method comprising:
sequentially positioning the mask relative to a drill whereby sequential vents in the array are drilled through the mask;
measuring a change in flow of a gas through the mask as the vents are drilled.

14. The method of claim 13, wherein the mask is positioned relative to the drill using a positioning system.

15. The method of claim 14, comprising a preliminary step of placing the mask on a support of the positioning system, the support having a surface configured to receive the mask in an airtight manner.

16. The method of claim 14, wherein the positioning system moves the mask relative to the drill.

17. The method of claim 13, wherein the vents are drilled through the mask in directions that vary from being normal to the mask.

18. The method of claim 13, wherein the vents are drilled using a laser drill.

19. The method of claim 13, wherein at least some of the vents are micro holes.

20. (canceled)

21. The method of claim 13, wherein a measured flow rate of the gas through the mask is determinative of a final configuration of the array.

22. The method of claim 13, wherein the array defines an indicia on the mask.

23. A mask produced using the apparatus of claim 1.
